(19) United States
(12) Patent Application Publication
(10) Pub. No.: US 2026/0116931 A1
(43) Pub. Date: Apr. 30, 2026

(54) IMPROVED ICE-BINDING PROTEINS BASED ON TWIST CONSTRAINED HELICES

(71) Applicants: Wageningen Universiteit, Wageningen (NL); Technische Universiteit Eindhoven, Eindhoven (NL)

(72) Inventors: Robbert Jan DE HAAS, Utrecht (NL); Rindert Jakob DE VRIES, Doorwerth (NL); Roderick TAS, Utrecht (NL); Ilja VOETS, Waalre (NL)

(21) Appl. No.: 19/118,702

(22) PCT Filed: Oct. 5, 2023
(86) PCT No.: PCT/NL2023/050521

§ 371 Date: Apr. 4, 2025

(30) Foreign Application Priority Data
Oct. 6, 2022 (EP) 22200103.4

(51) Int. Cl. C07K 14/46; A01N 1/125; A23B 2/85; C09K 3/18; C12N 1/20; C12N 15/70; C12R 1/19

(52) U.S. Cl. CPC C07K 14/461; A01N 1/125; A23B 2/85; C09K 3/185; C12N 1/20; C12N 15/70; C12R 2001/19

(57) ABSTRACT

The invention relates to a protein comprising at least one ice-binding alpha helix, wherein the ice-binding helix has a twist of less than 100 degrees per residue, preferably a twist of less than 99 degrees per residue, most preferably a twist of about 98.2 degrees per residue. The invention further relates to a composition comprising such protein. The invention further relates to the use of said protein or composition as a cryopreservation agent, as a gas hydrate inhibitor or in coatings for de-icing materials. Lastly, the invention furthermore relates to a method for cryopreserving an aqueous mixture using said protein and a method for producing said protein.

### FIELD

The invention relates to proteins having ice recrystallization inhibition activity, a high thermal stability and an efficient production method. Furthermore, the invention relates to the use of such proteins in methods for preventing or inhibiting ice recrystallisation in aqueous mixtures.

### 1 INTRODUCTION

To successfully bio-bank cellular materials, freezing and thawing rates of samples are carefully tuned in the presence of high concentrations of classic cryo-protective agents (CPAs) such as salt, DMSO, and glycerol to allow cells to dehydrate and prevent the formation of damaging intracellular ice (Pegg, 2002. Semin Reprod Med 20:5-13). Even in the presence of CPAs, extra-cellular ice that may form during the freezing process can induce mechanical damage by forming sharp ice-crystals that puncture cell membranes (Pegg, 2002. Semin Reprod Med 20:5-13). Additionally, long exposure of cellular materials to high concentrations of CPAs can lead to significant toxicity (Pegg, 2002. Semin Reprod Med 20:5-13). Extra-cellular ice crystallization and CPA toxicity are particularly problematic when attempting to freeze relatively large biological samples such as tissue biopts and organs. Compared to cell suspensions, freezing and thawing of large tissues must occur much slower and therefore large tissues may be exposed for a relatively long time to damaging CPAs. In part due to CPA toxicity, so far only ovaries (Campbell et al., 2014. Hum Reprod 29:1749-1763) and thymus slices (Ross et al., 2018. Eur J Immunol 48:716-719) have been successfully freeze-thawed without significant damage.

Ice binding proteins (IBPs) are a promising alternative to classic CPAs. These proteins are found in many organisms surviving at sub-zero temperatures where they influence growth, shaping, and nucleation of ice crystals. IBPs inhibit ice recrystallization at micromolar-millimolar concentrations to prevent freeze damage (Olijve et al., 2016. PNAS 113:3740-3745). Because of the low working concentrations and bio-compatibility of these protein materials, IBPs may (partially) substitute classic CPAs to mitigate risks of toxicity (Voets, 2017. Soft Matter 13:4808-4823). Moreover, adsorption of IBPs to extracellular ice crystals may help to prevent these crystals from shaping into damaging needle-like crystals and, thereby, to reduce damage to cell membranes during freezing and thawing.

Natural IBPs from plants, animals and bacteria can have a wide range of different activities such as preventing growth of ice-crystals and lowering the freezing temperature of water. These activities are starting to be exploited in industrial applications. For example, ice-binding proteins are being used to prolong the shelf life of ice-creams by preventing growth of ice-crystals (U.S. Pat. No. 6,914,043 B1) and to reduce the temperature required for producing artificial snow by lowering the temperature for ice-crystals to form (WO1983003831A1).

Natural IBPs display a wide diversity of structures. Many natural IBPs have an alpha-helical structure, some have a beta-solenoid structure, others are intrinsically disordered and heavily glycosylated (Bialkowska et al., Biomolecules 10:274). The enormous structural diversity of IBPs is one factor hampering the understanding of the activities of IBPs at a molecular-level. Another complicating factor is that the activities of IBPs cannot just be explained by their ice-binding activity and ice-binding specificity alone.

While structure-activity relationships are still obscure for many IBPs, their molecular geometry when binding to a specific ice-plane (i.e. the ice crystal surface) is starting to be elucidated, in particular through molecular simulation. One of the best studied natural IBPs is the winter flounder anti-freeze protein (wfAFP). It is one of the members of the so-called type-I class of IBPs. The wfAFP protein is a 37 residue alanine-rich almost straight alpha-helix (FIG. 1A) which is thought to bind especially to the pyramidal plane of ice and thus shape ice into characteristic bipyrimidal crystals. Helical type-I IBPs such as wfAFP have characteristic 11-mer consensus amino acid repeats of the general sequence TXXXAXXXAXX, where X is often alanine. It is proposed that wfAFP can bind to the plane of ice, since the 16.5 Å oxygen spacing on the ice plane very closely matches the threonine spacing of 16.7 Å of the 11-mer consensus repeat (Sicheri et al., 1995. Nature 375:427-431; Jia et al. 2002. Trends Biochem Sci 27:101-106). Threonines on wfAFP can be altered into valines without loss of activity, but altering them into serines abolishes activity suggesting that hydrophobic interactions at those positions are crucial (Chakraborty et al., 2017. Phys Chem Chem Phys 19:11678-11689).

Several attempts have been made to leverage the unique properties of natural IBPs to reduce freeze-thaw damage in biological samples, but thus far with limited success (Tomas et al., 2019. Biomacromolecules 20:3864-3872). A major bottleneck to their application is the thermal instability of IBPs, which makes them prone to aggregation in vivo/in situ. For example, many natural IBPs lose their function when exposed to room temperature. Due to their thermal instabilities, IBPs are also difficult to engineer to tune their activity. Moreover, natural IBPs are often difficult to purify recombinantly, making the production of natural proteins expensive compared to traditional CPAs.

There is need for improved strategies for the cryo-preservation of sensitive materials. Especially, novel methods and means are required to freeze-thaw cells and especially larger tissue samples for bio-banking of biopts and/or (parts of) donor organs such as the liver, pancreas, kidney and the heart. There is thus a need for the development of new cryo-preservation agents that are effective in the inhibition of ice recrystallization, that are thermally stable and that can efficiently be produced.

### 2 BRIEF DESCRIPTION OF THE INVENTION

It was found that an artificial ice-binding protein (aIBP) with high thermal stability and high ice recrystallization inhibition (IRI) activity can be de novo designed and obtained by expression in a host cell. Such aIBP comprises at least one ice-binding alpha helix and one or more stabilizing alpha helices. Such artificial protein possesses a specific twist in an ice-binding alpha helical structure, also observed in natural IBPs. The specific twisting of the alpha helical structure resulted in a precise positioning of amino acid residues within the twisted helix, in particular of threonines, allowing the protein to precisely bind to an ice crystal lattice. It was furthermore found that the twisting of such helical structures can be stabilized by one or more stabilizing alpha helices. Said one or more stabilizing helices differ from the ice-binding alpha helical structure. The binding of an aIBP to ice crystals was found to decelerate and even stop further growth of the crystals, and to shape the crystal in predominantly blunt-end shapes. The artificial proteins were found to possess high thermal stability and high IRI activity. They can be used as additive to prevent freeze-thaw damage in biological materials or food products. Other applications of these artificial proteins include their use as gas hydrate inhibitor, which helps to avoid the formation of hydrate plugs and line blockages during natural oil or gas production, and their use in coatings for de-icing materials such as plane wings, window shields or structures of wind turbines (such as blades). Importantly, said aIBPs are thermostable and can be efficiently produced using biotechnological production processes.

Therefore, the invention provides a protein comprising at least one ice-binding alpha helix and one or more stabilizing alpha helices, wherein the ice-binding helix has a twist of less than 100 degrees per residue, preferably a twist of less than 99 degrees per residue, most preferably a twist of about 98.2 degrees per residue. Said one or more stabilizing alpha helices differ from the at least one ice-binding alpha helix, for example in their amino acid sequences.

Preferably, said at least one ice-binding alpha helix comprises at least two copies of sequence TXXXAXXXAXX, preferably at least three copies of sequence TXXXAXXXAXX, wherein T represents a threonine residue, A represents an alanine residue and X represents any amino acid residue. More preferably, the at least one ice-binding alpha helix comprises at least two copies of sequence TXAXAXLXAX[I/L]V, preferably at least three copies of sequence TXAXAXLXAX[I/L]V, wherein T represents a threonine residue, A represents an alanine residue, I represents an isoleucine residue, L represents an leucine residue, V represents an valine residue and X represents any amino acid.

Preferably, the one or more stabilizing alpha helices are linked to the at least one ice-binding alpha helix.

A protein according to the invention is thermally stable such that the structure of the alpha helix is remained at temperatures above 20° C., preferably at temperatures above 30° C., preferably at temperatures above 65° C., preferably at temperatures above 75° C., preferably at temperatures above 85° C., most preferably at temperatures above 95° C.

In a protein according to the invention, preferably the total number of amino acid residues of the protein is between 33 and 350 amino acid residues, preferably between 100 and 200 amino acid residues, most preferably about 136 amino acid residues.

A preferred protein according to the invention comprises a sequence having at least 80% sequence identity with any one of SEQ NO: 1-8.

Furthermore, the invention provides a composition comprising an effective amount of a protein according to the invention and one or more other components selected from water, DMSO, glycerol, trehalose, fetal calf serum, cell culture medium, a buffer, an antibiotic, an anti-coagulant, an anti-oxidant and a pH indicator.

Furthermore, the invention provides the use of a protein or composition according to the invention as a cryopreservation agent, as a gas hydrate inhibitor or in coatings for de-icing materials such as aircraft wings, drones, air conditioners, refrigerators, freezers, electricity cables, window shields or structures of wind turbines such as blades.

Further, the invention provides a method of stabilizing an ice-binding alpha helix, comprising the steps of: (a) providing an ice-binding alpha helix; and (b) linking one or more different alpha helices to the ice-binding alpha helix of the protein; thereby stabilizing the ice-binding alpha helix.

Further, the invention provides a method for cryopreserving an aqueous mixture, comprising contacting the aqueous mixture with a protein or composition according to the invention. In said method, the aqueous mixture may be a biological material a tissue, an organ, or part thereof. In said method, the aqueous mixture may be a food product such as ice cream, meat, a fruit or a vegetable.

Further, the invention provides a method for producing a protein according to the invention, comprising the steps of: (a) providing an expression vector comprising a nucleic acid encoding a protein according to the invention in a host cell, preferably said host cell is a bacterium, most preferably Escherichia coli; (b) expressing the protein; and (c) optionally purifying the protein from the host cell.

### 3 BRIEF DESCRIPTION OF THE FIGURES

FIG. 1. Structure of the winter flounder anti-freeze protein (wfAFP, protein data bank (pdb) id: 1WFA). (A) The wfAFP shows a simple alpha-helical ice binding protein fold. (B) The wfAFP shows a twisting of its helix of 98.2° (ω1), which is 1.8° less than an idealized helix) (ω1=100°). (C) Rosetta energy (reu) of straight 44 residue helix with sequence [TAAAAAAAAA]₄, where T is threonine, A is alanine at different twistings, demonstrating the ideal per residue twisting of a helix is between 100-101°. (D) Examples of straight helices from panel C, aligned in the z-axis, with threonine residues displayed as sticks showing that under-twisting of a perfect helix of ω1=100° to ω1=98.2° leads to perfect alignment of threonine residues.

FIG. 2. Structure of the sculpin antifreeze protein (pdb: 1Y03). Similar to wfAFP (FIG. 1). (A) The sculpin AFP shows a simple alpha-helical ice binding protein fold and (B) shows a twisting of its helix of 98.2° (ω1), which is 1.8° less than an idealized helix (ω1=100°).

FIG. 3. Stabilisation strategy of de novo ice binding proteins. Three-fold cyclic symmetry group (C3) helical bundles with ice-binding residues on one helix (shown in dark grey) were parametrically sampled and each backbone was designed using the Rosetta protein design software (with fixed backbone design). Chains of select designs were closed by loop connection, which were amino acid residues loops of 2 to 3 residues long.

FIG. 4. Experimental validation of the TIP-98 design. (A) SDS-PAGE gel showing various stages of protein purification, and showing high purity and yield obtained after expression in E. coli. Lane 1: Sample post-induction with isopropyl B-D-thiogalactoside (IPTG), lane 2: lysate after sonication, lane 3: supernatant after centrifugation of the lysate, lane 4: flow-through of supernatant on the affinity column, lane 5: eluate from the affinity column of the purified protein. (B) Size-exclusion chromatography (SEC) showing the protein is monomeric. Y-axis shows normalized absorbance at 280 nm and x-axis show the retention volume (rv) of the column used (Superdex 75 10/300 gl, GE Healthcare). (C) Circular dichroism (CD) shows protein stability at 20° C., 95° C. and refolding at 20° C. after heating. Y-axis shows molar ellipticity and x-axis shows wavelength. (D) Molar ellipticity (θ) at 222 nm during temperature ramp from 20° C. to 95° C. at 1° C./min. (E) Energy landscape from Rosetta Ab initio folding simulations. Each datapoint represents a protein model output from an independent folding trajectory. Y-axis shows the energy “score” of the structure quantified using the Rosetta energy score function (REF2015) in rosetta energy units (reu). X-axis shows the Ca root mean square deviation (rmsd) relative to the design in Å. A single funnel towards rmsd <1.5 Å shows the designed model has a single global energy minimum.

FIG. 5. Ice recrystallization inhibition over time. (A) Ice crystal growth over time is reduced in the presence of TIP-98 design at various concentrations as indicated. In the assay, a first phase with mainly rapid coalescence (“fusion events”) was followed by a second slower phase of mainly Ostwald ripening (“ripening”). A decrease in ice crystal growth can be observed when comparing the samples comprising various concentrations of the TIP-98 design to the 0 μM control. (B) Ice crystal volumes were quantified as a function of time and TIP-98 concentration. In brief, 8-bit images of the ice-crystals were subjected to the bandpass filter, enhance contrast and subtract background function of ImageJ. Subsequently, the bright signal at the edges and then the individual crystals were isolated by the autoThreshold and Convert to Mask function. Analyze Particles was used to obtain the area of each crystal. The data was imported into Matlab upon which the radius (r) was calculated in order to determine the corresponding spherical volume of each crystal. A smaller slope, indicative for a lower growth rate, is observed when concentrations of the TIP-98 design increase. (C) Recrystallization growth rates (ka) were determined by applying a linear fit to the resulting ice-volume as a function of time traces.

FIG. 6. Ice shaping in the presence of the TIP-98. TIP-98 induces ice crystal shaping upon decreasing temperatures. Temperature was gradually decreased from −7° C. to −11° C. in 2 minutes. Protein adsorption to certain planes on the ice crystal lead to retarded growth and therefore shaping of the final crystals. At concentrations >20 μM mostly blunt-end and occasional bipyrimidal ice crystals are observed.

FIG. 7. Mutant proteins with enhanced ice-recrystallisation inhibition (IRI) activity. (A) A side view of the TIP-98 design, two mutants TIP-98^{2A} and TIP-988A and tandem mutant TIP-98^{2A8A} are shown, with the top helix being the ice-binding helix. (B) The 11 residue consensus sequence of the TIP-98 ice-binding helix is shown. 2A and 8A designations mean substitution on position 2 and 8 in the 11-mer consensus sequence. (C) Plotting the ice crystal growth (k₁) as function of TIP concentration (c) for the TIP-98 and three mutants, designs shows enhanced activity (i.e. slower crystal growth) at lower concentrations for the mutant designs.

FIG. 8. Computational helical bundle structure. (A) Computationally obtained structure for a helical bundle TIP with a helix-loop-helix-loop-helix structure (indicated by H1-L1-H2-L2-H3) with a central ice binding helix H2 where the minimal ice-binding consensus sequence is indicated with amino acids represented as sticks. The helices consists of 4 repeats (rep), where the edges are defined as two halve repeats (0.5 rep) and the middle contains 3 full repeats. (B) Computationally obtained structures demonstrate tight core packing of hydrophobic amino acid residues in 11-mer sequences. The spatial arrangement of the active ice-binding amino acid residues is maintained in the same spatial arrangement as in the natural template, in this case the winterflounder type I AFP.

FIG. 9. Crystal structure of the TIP-99a design. (A) Cartoon diagram showing TIP-99a crystal structure (light gray) and Rosetta relaxed design model (dark gray) with an overall backbone RMSD of 1.12 Å. The crystal structure of TIP-99a was solved at 2.3 Å. The helical twisting of the ice-binding helix and the ice-binding residues are maintained at 99.2° per residue with a 0.67 Å backbone RMSD of ice-binding helix. (B) Three inserts at different locations in the helical bundle showing rotamer packing in the crystal structure, particularly in the core, highly match with the designed model.

### 4 DETAILED DESCRIPTION OF THE INVENTION

### 4.1 Definitions

As are used herein, the singular forms “a”, “an” and “the”, are intended to include the plural forms as well.

As is used herein, the term “or” includes any and all combinations of one or more of the associated listed items, unless the context clearly indicates otherwise (e.g. if an “either .or” construction is used).

As are used herein, the terms “comprise” and “comprising”, and conjugations thereof, are open language and specify the presence of stated features but do not preclude the presence or addition of one or more other features.

It will be understood that when a particular step of a method is referred to as subsequent to another step, it can directly follow said other step or one or more intermediate steps may be carried out before carrying out the particular step, unless specified otherwise.

As is used herein, the term “protein” refers to an organic linear, circular, or branched polymer composed of two or more amino acid monomers and/or analogues thereof. A protein is usually composed of a linear chain of amino acid residues covalently linked by a peptide bond (CO—NH) or a synthetic covalent linkage. The amino acid monomers of a protein may be naturally occurring amino acid residues or non-naturally occurring amino acid residues. The term “protein” encompasses a native or modified protein, a protein fragment, a protein analogue comprising non-naturally occurring amino acid residues. A protein may be monomeric or polymeric. The amino acid sequence of a protein may be one that occurs in nature or may be engineered. Protein sequences, as used herein, are a linear representation of the amino acid residues of a protein in an amino-terminal (i.e. N-terminal) to carboxy-terminal (i.e. C-terminal) direction. Abbreviations of the standard, naturally occurring, amino acid residues, as used herein, include alanine (A), cysteine (C), aspartic acid (D), glutamic acid (E), phenylalanine (F), glycine (G), histidine (H), isoleucine (I), lysine (K), leucine (L), methionine (M), aspartic acid (N), proline (P), glutamine (Q), arginine (R), serine(S), threonine (T), valine (V), tryptophan (W), and tyrosine (Y).

As is used herein, the term “alpha helix” or “a helix”, refers to a right-hand-coiled or spiral conformation (i.e. helix) of a protein or part of a protein. Usually in an alpha helix, every backbone N—H group donates a hydrogen bond to the backbone C═O group of the amino acid three or four residues earlier (i.e. more towards the N-terminus of the protein). The alpha helix is a common secondary structure of proteins. The most common alpha helix in nature is the so called “Pauling-Corey-Branson α-helix” or “3.6₁₃-helix”. Such helix has an average number of residues of about 3.6 per one helical turn meaning that each amino acid residue corresponds to a 100 degree turn in the helix (i.e. the “helical twist”, expressed as 100 degrees per residue). The 13 in the term “3.6₁₃-helix” refers to the 13 atoms that are involved in the ring formed by the hydrogen bond.

As is used herein, the term “under-twisted helix”, refers to a helix having a smaller twist as observed in the classical 3.6₁₃-helix. More specifically, an under-twisted helix has a twist of less than 100 degrees per residue, such as a twist of 98.2 degrees per residue.

As is used herein, the unit “Angstrom (Å)”, refers to a unit of length equal to 0.1 nanometer (nm). Angstrom is used to indicate the distance along the helix axis. For example, in a 3.6₁₃-helix, the distance between consecutive turns of the helix is 5.4 Å (or 0.54 nm).

As is used herein, the term “protein bundle” or “helix bundle”, refers to a protein comprising two or more helices. Usually these helices are symmetrically organised such as nearly parallel or antiparallel to each other. An example of a protein bundle is a protein comprising three alpha helices organised in a 3-fold cyclic symmetry group (C3), such as collagen.

As is used herein, the term “ice-binding protein (IBP)”, refers to a protein capable of binding to an ice crystal so as to inhibit growth and recrystallization of the ice crystal. IBPs can be classified into four types according to their structure (Davies and Hew, 1990. FASEB J 4:2460-2468). The type I IBPs are alanine-rich with regularly spaced threonine and/or asparagine residues and have an α-helical conformation. Type II IBPs have a characteristic high cysteine content (about 8%). Type III IBPs are small globular peptides of approximately 64 amino acid residues long. Type IV IBPs have a repeated tripeptide motif to which is attached a disaccharide. Traditionally, the term “ice-binding protein (IBP)” was considered a synonym of the term “antifreeze proteins (AFPs)”, referring to protein having two main activities being ice recrystallization inhibition and thermal hysteresis. As used herein, the term “ice-binding protein (IBP)” refers to a diverse group of proteins, comprising antifreeze protein (AFP) and ice nucleation protein (INP), having ice binding capabilities.

As is used herein, the term “binding” in the context of the binding of a protein to an ice crystal, refers to a binding with high affinity, such as an affinity corresponding to a low IC50 value, such as an IC50 below 1 mM.

As is used herein, the term “IC50”, refers to the half-maximum mean inhibitory concentration, which is a quantitative measure indicating the concentration of a substance, here an IBP, to inhibit a specific biological or biochemical function, here the crystal growth rate. The IC50 of an IBP can be determined by measuring the ice crystal radius during freezing in the presence of various concentrations of IBP, and calculating the average volume growth rate over time.

As is used herein, the term “ice recrystallization”, refers to the phenomenon observed as an increase in ice crystal size within a frozen mixture or partially frozen mixture. Usually during ice recrystallization, the increase in crystal size of large crystals is at the expense of smaller ones so as to minimize the total surface energy of the system. Ice recrystallization occurs due to cooling conditions in a partially frozen environment or due to temperature fluctuations within a frozen material. In addition to an increase in ice crystal size, ice recrystallization may also result in the formation of sharper crystals that damage materials such as cell membranes. Ice recrystallization can be detrimental to many materials and products. In cryopreservation of food products, such as for example ice cream, ice recrystallisation can cause a loss of soft texture and deterioration of quality during storage. In cryopreservation of a biological material, ice recrystallization during freezing or thawing may be harmful for cells and tissues, as it may damage cell membranes and promotes cell dehydration.

As is used herein, the term “ice grain boundary” refers to the juncture between individual ice crystals (also termed “grains”) in a crystal structure.

As is used herein, the term “ice recrystallisation inhibition (IRI)”, refers to the inhibition of ice recrystallization and thus the maintaining of small sized ice crystals within a frozen mixture or partially frozen mixture. A mixture with high IRI activity can minimise or even prevent ice crystal growth. Such mixtures can therefore be used for cryopreservation and in cryopreservation compositions.

As is used herein, the term “ice-binding helix”, refers to the helix of an ice-binding protein, such as a Type I IBP, that is capable of binding to an ice crystal.

As is used herein, the term “cryopreservation”, refers to the preservation of a mixture at a temperature below 4° C. Preferably, the cryopreservation temperature is below 0° C., such as below −5° C., −10° C., −20° C. or −60° C. Cryopreservation can be obtained by quick freezing of a mixture so that ice crystals are too small to rupture cells, for example by using liquid nitrogen or carbon dioxide. Liquid nitrogen or carbon dioxide result in the preservation of a mixture at a temperature of about-196° C., or −80° C., respectively.

As is used herein, the term “freezing”, refers to reducing the temperature to a cryopreserving temperature. The term “frozen” refers to the state of a mixture at such temperature.

As is used herein, the term “quick freezing” or “flash freezing”, refers to freezing in a relatively short period of time. Quick freezing can be performed by contacting a mixture with, for example, carbon dioxide (about −80° C.), liquid nitrogen (about −196° C.), or liquid helium (about −269° C.).

As is used herein, the term “preservation of a biological material”, refers to the process of maintaining biological material under conditions in which its biological activity is considerably reduced while it nonetheless remains viable and may resume essentially normal biological activity when taken out of the preservation state.

As is used herein, the term “preservation of a food product”, refers to the process of maintaining a food product under conditions in which the quality of the food product is not substantially affected. Factors determining the quality of food that may be affected by preservation include, but are not limited to, product shrinkage, toughening, loss of texture, product shelf life, microbial activity and dehydration loss.

As is used herein, the term “aqueous mixture” refers to a mixture comprising significant quantities of water such as e.g. at least 5%, at least 10% or at least 20% water by weight. An aqueous mixture is susceptible to ice crystal growth upon cryopreservation and/or thawing therefrom. A preferred aqueous mixture is a biological material or a food product.

As is used herein, the term “biological material”, refers to a liquid, solid or semisolid product that includes at least one cell, tissue, whole organ or part of an organ.

As is used herein, the term “cell”, refers to a bacterial cell, fungal cell, plant cell, animal cell, preferably mammalian cell, and most preferably human cell. A preferred cell is a sperm cell, an ovum, a stem cell, a muscle cell, a heart cell, a brain cell and/or a blood cell.

As is used herein, the term “cell-containing animal product”, refers to a component derived, isolated and/or purified from an animal's body including a cell, tissue, whole organ and part of an organ.

As is used herein, the term “cell-containing plant product”, refers to a component derived, isolated and/or purified from a plant including a cell, tissue, or plant part such as pollen, ovule, leave, embryo, root, root tip, anther, flower, fruit, stem, shoot, scion, rootstock, seed, protoplast, callus, and the like.

As is used herein, the term “tissue”, refers to an aggregate of cells that together perform certain special functions. The term includes reference to a biopsy, a skin graft, a cornea, a section of an artery or vein, ovarian tissue, a tissue slice and/or a transplant tissue. A preferred tissue is an animal tissue, including a human tissue, or a plant tissue such as a seed.

As is used herein, the term “organ”, refers to a differentiated structure that comprises cells and/or tissues and performs a specific function in an organism. The term includes reference to a kidney, heart, lung, spleen, pancreas and/or liver. A preferred organ is an organ from an animal, including human.

As is used herein, the term “food product”, refers to a mixture that is usually composed of carbohydrates, fats, proteins and water, and which can be eaten or drunk by any animal including humans. Such food product may be a frozen product such as ice cream, frozen yoghurt or sorbets, or may be frozen during storage until consumption such as meat, a fruit or a vegetable. A food product may benefit from a reduction or inhibition of ice crystal growth, for example during production and/or storage.

As is used herein, the term “composition comprising an effective amount of an aIBP”, refers to a composition comprising a specific quantity of a protein according to the invention in order to reduce or inhibit growth and recrystallization of an ice crystal. Amounts effective to achieve said reduction or inhibition of growth and recrystallization of an ice crystal will depend on the application. Said composition can be used as cryopreserving composition and is suitable for the preservation of biological material and food products. A composition according to the invention may further comprise at least one of water, DMSO, glycerol, trehalose, cell culture medium, a buffer, an antibiotic, an anti-coagulant, an anti-oxidant and a pH indicator.

As is used herein, the term “substance” refers to a material which is of a particular kind or constitution. The term substance includes reference to a solid surface onto which the formation of ice crystals is to be reduced or inhibited. Such solid surface includes the wings of an airplane or windmill, tail surfaces of an airplane and the blades of a propeller.

As is used herein, the term “contacting a mixture” refers to the action of bringing a mixture such as an aqueous mixture into contact with a protein or composition according to the invention. In a method of cryopreserving a mixture, the contacting of the mixture with a protein or composition according to the invention may occur prior to and/or during cryopreservation. Preferably, the mixture is contacted prior to cryopreservation.

As is used herein, the term “contacting a substance” refers to the action of bringing a substance into contact with a protein or composition according to the invention. In a method of cryopreserving a substance, the contacting of the substance with a protein or composition according to the invention may occur prior to and/or during cryopreservation. Preferably, the substance is contacted prior to cryopreservation.

As is used herein, the term “thermal stability of a protein”, refers to the ability of a protein to resist a change in structure due to a difference in temperature. For example, when a protein is heated to a temperature above a threshold temperature, thermal energy may cause unfolding and denaturation of the protein. A protein that is capable of withstanding a high temperature, such as a temperature above 20° C., or above 30° C., preferably above 65° C., is termed a protein with a high thermal stability. There are various methods known to a skilled person to determine the thermal stability of a protein including, but not limited to, circular dichroism (CD), X-ray crystallography, electron crystallography and nuclear magnetic resonance spectroscopy (NMR) spectroscopy.

As is used herein, the term “gas hydrate inhibitor”, refers to a protein or composition comprising a protein that is able to prevent or retard the formation of gas hydrates, or reduce the tendency for said hydrates to agglomerate during storage and/or hydraulic transport of hydrocarbon-based fluids comprising water.

The term “vector”, as is used herein, refers to a nucleic acid molecule capable of transporting genetic material to which it has been linked, or which is incorporated into the vector. The term vector includes, but is not limited to, a nucleic acid molecule that is single-stranded, double-stranded, or partially double-stranded; a linear or circular nucleic acid molecule; a nucleic acid molecule that comprise DNA, RNA, or both; and a combination and other varieties of a nucleic acid molecule known in the art. A vector is often used to transduce a gene encoding a protein of interest into a suitable host cell. Once in the host cell, the vector may replicate independently of, or coincidental with, the host chromosomal DNA. Examples of commonly used vectors are plasmids, viral vectors such as retroviral vectors, and bacteriophage-related vectors such as based on the Escherichia coli M13 phage.

The term “expression vector”, as is used herein, refers to a vector that is able to direct expression of one or more genes to which they are operatively-linked. Suitable regulatory elements include promoters, enhancers, internal ribosomal entry sites (IRES), and other expression control elements such as 5′ untranslated regions, optionally containing a ribosome binding site, 3′ untranslated region optionally comprising a ‘post stop-codon, ante terminator’ region, terminator sequences, and transcription termination signals such as polyadenylation signals and poly-U sequences.

The term “capping”, or variants thereof such as capped, as is used herein, refers to the covering, or protecting, of a free end of a protein. Capping may be performed by the modification of one or more ends of a protein, or by the addition of one or more amino acid residues, such as 2-8 amino acid residues, including 5 and 6 amino acid residues, that function to protect degradation of the protein.

### 4.2 Ice Binding Alpha Helix

The ice-binding activity of a protein arises from a combination of the chemical identity of the amino acid residues contacting the ice, as well as their precise spatial arrangement with respect to the ice plane to which they bind. The spatial arrangement of the ice-binding amino acid residues is determined by a combination of sequence and structure of the protein in the vicinity of the ice-binding residues, such as secondary and tertiary structure of the protein. Chemical identities of amino acid residues and suitable spatial arrangements for ice-binding activity can be inferred from natural examples for which both structure and activity are known, for example winter flounder AFP (wfAFP).

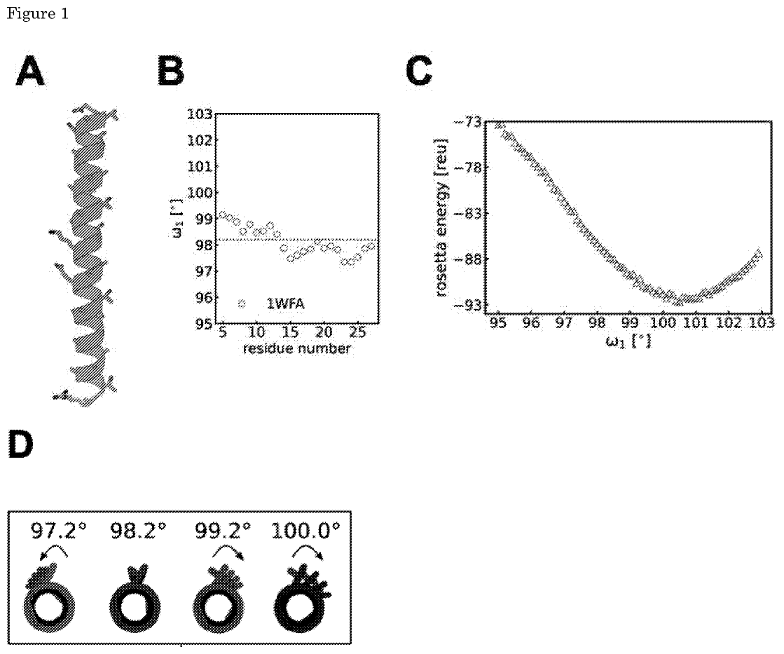

It is known that helical type I IBPs such as wfAFP have characteristic repeats of an 11-mer with sequence TXXXAXXXAXX, wherein T represents a threonine residue, A represents an alanine residue and X represents any amino acid. Said characteristic repeat is repeated three times in the sequence of wfAFP. In order to have ice recrystallization inhibition (IRI) activity the helical structure of the IBPs must be under-twisted. It was found that, in contrast to the usual straight alpha helices that show a twist of 100 degrees per residue, the alpha helix of an IBP such as wfAFP shows a helical twist of 98.2 degrees per residue. It appears due to this under-twisting that the threonines of the 11-mer sequence all point to the same direction (see FIG. 1D), which is required for the IBP to bind an ice crystal.

Therefore, the invention provides a protein comprising at least one ice-binding alpha helix. Said ice-binding helix is an alpha helix which is under-twisted compared to a general alpha helix having a twist of 100 degrees per residue. Said ice-binding helix has a twist less than 100 degrees per residue such as a twist of between 96.5 and 99.2 degrees per residue, preferably a twist of less than 99 degrees per residue such as a twist of between 97 and 98.9 degrees per residue, more preferably a twist of between 97.5 and 98.5, most preferably a twist of about 98.2 degrees per residue or a twist of exactly 98.2 degrees per residue.

A preferred ice-binding alpha helix comprises at least two copies of sequence TXXXAXXXAXX, preferably at least three copies of sequence TXXXAXXXAXX, wherein T represents a threonine residue, A represents an alanine residue and X represents any amino acid, and has a twist of about 98.2 degrees per residue. The number of copies of the sequence TXXXAXXXAXX is 3 or more, such as between 3 and 10, preferably 3, 4, 5, 6, 7, 8, 9 or 10. In every copy of the consensus sequence TXXXAXXXAXX, X may be independently chosen. X may refer to the same or similar amino acid residue in some copies. In some embodiments, every sequence of the consensus TXXXAXXXAXX is identical in an ice-binding alpha helix according to the invention.

The twisting of an ice-binding alpha helix according to the invention is such that the threonines of the at least two or the at least three sequences TXXXAXXXAXX, as comprised within an ice-binding alpha helix according to the invention, all point to the same direction in an ice-binding alpha helix according to the invention. A preferred ice-binding alpha helix comprises at least two copies of TXXXAXXXAXX, preferably at least three copies of TXXXAXXXAXXX, wherein every copy is independently selected from sequence TXAXAXLXA[I/L]V, TAAXAXLXA[I/L]V, TXAXAXLAA[I/L]V, or TAAXAXLAA[I/L]V. A more preferred ice-binding alpha helix comprises at least two copies of TXXXAXXXAXXX, preferably at least three copies of TXXXAXXXAXXX, wherein each copy has the sequence TXAXAXLXA[I/L]V, TAAXAXLXA[I/L]V, TXAXAXLAA[I/L]V, or TAAXAXLAA[I/L]V.

An ice-binding alpha helix may comprise any one of sequences SEQ NO: 1-4. Proteins comprising such ice-binding alpha helix were thermal stable and showed good results in terms of IRI activity. Therefore, a preferred ice-binding alpha helix of the invention comprises three copies of the consensus TXXXAXXXAXX with a sequence having at least 80% sequence identity, preferably 90% sequence identity, most preferably 100% sequence identity with any one of sequences SEQ NO: 1-4.

### 4.3 Stabilizing an Ice Binding Alpha Helix

To stabilize the ice-binding alpha helix in an IBP according to the invention, said protein may further comprise one or more stabilizing alpha helices, such as two stabilizing helices or three stabilizing helices. These one or more stabilizing helices may be linked to the ice-binding alpha helix to stabilize its twisting. Said linkage may be provided by a covalent interaction between a stabilizing helix and an ice-binding helix, such as a direct linkage of both helices via a peptide bond or a linkage via peptide bonds with a loop between both helices.

A loop linking an ice-binding helix to a stabilizing helix preferably is a short amino acid sequence comprising between 1 and 10 amino acid residues, such as 2, 3, 4, 5, 6, 7, 8, or 9 amino acid residues. Said loop preferably comprises 2 amino acid residues. Said loop may link the N-terminal part of an ice-binding helix to the C-terminal part of a stabilizing helix, and/or the C-terminal part of an ice-binding helix to the N-terminal part of a stabilizing helix. As is shown in the examples, IBPs comprising an ice-binding alpha helix and two stabilizing alpha helices organised in a straight helical bundle showed good IRI activity and thermal stability.

Alternatively, said linkage may be provided by a non-covalent interaction between a stabilizing helix and an ice-binding helix such as e.g. an electrostatic interaction or an interaction based on hydrogen bonding and/or Van der Waals forces.

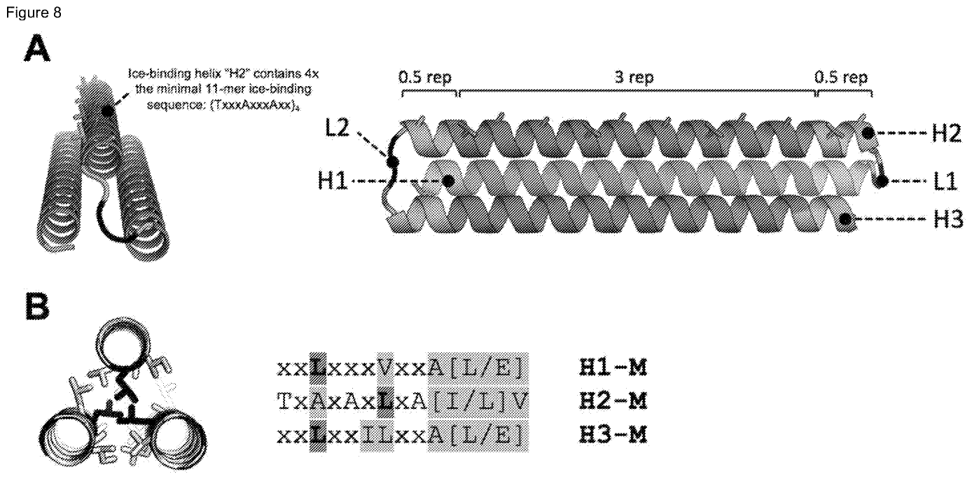

The sequence architecture of a preferred protein according to the invention comprising an ice-binding alpha helix and two stabilizing alpha helices, may have a “Helix-Loop-Helix-Loop-Helix” sequence architecture represented as H1-L1-H2-L2-H3, wherein H2 is the central ice-binding helix featuring at least three copies of the ice-binding motif TXXXAXXXAXX, H1 and H3 are stabilizing alpha helices and L1 and L2 are loop sequences (FIG. 8A).

Preferably a helix according to the invention, such as an ice-binding helix or stabilizing helix, is capped with a cap sequence. The advantage of capping is the reduction of the flexibility of the helix edges. A flexible helix edge is undesired as it can distort proper helix formation and can distort twisting of the helix. Additionally, helix residues near the loop sequences have another chemical environment, e.g. more interactions with solvent or more interaction with loop residues, compared to the 11-mer motifs located in the middle of the helices.

Preferably, a protein according to the invention comprising an ice binding alpha helix and two stabilizing alpha helices, has a sequence architecture as provided by following formula (I):

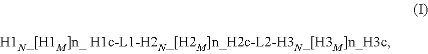

wherein:
H1_{N} depicts the N-terminal cap of helix 1;
H1_{C} depicts the C-terminal cap of helix 1;
H2_{N} depicts the N-terminal cap of helix 2;
H2_{C} depicts the C-terminal cap of helix 2;
H3_{N} depicts the N-terminal cap of helix 3;
H3_{C} depicts the C-terminal cap of helix 3;
L1 depicts the loop sequence linking helix 1 to helix 2;
L2 depicts the loop sequence linking helix 2 to helix 3;
H1_{M} is an n-fold repetition of a 11-mer motif within helix 1;
H2_{M} is an n-fold repetition of the ice-binding motif TXXXAXXXAXX within helix 2;
H3_{M} represents an n-fold repetition of a 11-mer motif within helix 3;
N is an integer between 3 and 10.

Preferably, an N-terminal cap, such as H1_{N}, H2_{N} and/or H3_{N}, has a length of between 2 and 11 amino acid residues, more preferably between 4 and 8 amino acid residues, more preferably 5 or 6 amino acid residues, most preferably 5 amino acid residues.

Preferably, a C-terminal cap, such as H1_{C}, H2_{C} and/or H3_{C}, has a length of between 2 and 11 amino acid residues, more preferably between 4 and 8 amino acid residues, more preferably 5 or 6 amino acid residues, most preferably 6 amino acids.

Preferably, the sum of the lengths of the C-terminal cap and the N-terminal cap of a helix, such as the sum of the lengths of H1_{C} and H1_{N}, the sum of the lengths of H2_{C} and H2_{N} or the sum of the lengths of H3_{C} and H3_{N}, is 11 residues.

Preferably H2_{N} has a length of 5 amino acid residues and features motif XXAXX. Most preferably H2_{N} has a length of 5 amino acid residues and features motif XXATI.

Preferably H2_{C} has a length of 6 amino acid residues and features motif TXXXAX. Most preferably a H2_{C} has a length of 6 amino acid residues and features motif TXAXAX.

Preferably, a cap may be formed by the amino acid sequences selected from any one of XₚₒₗₐᵣXₚₒₗₐᵣXₚₒₗₐᵣAL, XₚₒₗₐᵣXₚₒₗₐᵣ LXₚₒₗₐᵣXₚₒₗₐᵣL, TXₚₒₗₐᵣAXₚₒₗₐᵣAXₚₒₗₐᵣ, TAAXₚₒₗₐᵣAXₚₒₗₐᵣ, XₚₒₗₐᵣXₚₒₗₐᵣATI, XₚₒₗₐᵣAATI, XₚₒₗₐᵣXₚₒₗₐᵣXₚₒₗₐᵣAL and XₚₒₗₐᵣXₚₒₗₐᵣLXₚₒₗₐᵣXₚₒₗₐᵣXₚₒₗₐᵣ for any one of H1_{N}. H1_{C}, H2_{N}. H2_{C}, H3_{N} and H3_{C}, wherein Xₚₒₗₐᵣ can be any polar, i.e. non-hydrophobic, amino acid selected from D, E, H, K, N, Q, R and S. Preferably, the cap sequences are sequences XₚₒₗₐᵣXₚₒₗₐᵣXₚₒₗₐᵣAL for H1_{N}. XₚₒₗₐᵣXₚₒₗₐᵣ LXₚₒₗₐᵣXₚₒₗₐᵣL for H1_{C}, XₚₒₗₐᵣXₚₒₗₐᵣATI or XₚₒₗₐᵣAATI for H2_{N}. TXₚₒₗₐᵣAXₚₒₗₐᵣAXₚₒₗₐᵣ or TAAXₚₒₗₐᵣAXₚₒₗₐᵣ for H2_{C}, XₚₒₗₐᵣXₚₒₗₐᵣXₚₒₗₐᵣAL for H3_{N} and XₚₒₗₐᵣXₚₒₗₐᵣLXₚₒₗₐᵣXₚₒₗₐᵣXₚₒₗₐᵣ for H3_{C}, wherein Xₚₒₗₐᵣ can be any polar, i.e. non-hydrophobic, amino acid selected from D, E, H, K, N, Q, R and S.

Preferably, a cap may be formed by the amino acid sequences selected from any one of EEEAL, EKLKKL, TEASAN, TAASAN, DEATI, DAATI, SEEAL and ERLDRN for any one of H1_{N}. H1_{C}, H2_{N}. H2_{C}, H3_{N} and H3c. Most preferably, the cap sequences are sequences EEEAL for H1_{N}. EKLKKL for H1_{C}, DEATI or DAATI for H2_{N}. TEASAN or TAASAN for H2_{C}, SEEAL for H3_{N} and ERLDRN for H3c.

Preferably, a loop sequence comprises two amino acid residues of which at least one is a G or P. Preferred loop sequences are selected from any one of GK and GV for any one L1 and L2, most preferably GK for L1 and GV for L2.

Preferred 11-mer motifs are selected from any one of XXLXXXVXXA[L/E] and XXLXXILXXA[L/E] for a stabilizing helix such as any one of HIM and H3M, most preferably XXLXXXVXXA[L/E] for HIM and XXLXXILXXA[L/E] for H3M.

Preferred ice-binding motifs as comprised in H2_{M} TXAXAXLXA[I/L]V, TAAXAXLXA[I/L]V, TXAXAXLAA[I/L]V and/or TAAXAXLAA[I/L]V.

Preferred elements of a protein according to the invention are provided in Table 1. A protein according to the invention comprising the sequences as provided in Table 1, was found to have tightly packed cores with the hydrophobic residues of the H1_{M}. H2_{M} and H3_{M} repeats maintaining the spatial arrangement of the ice-binding amino acid residues in the same spatial arrangement as in the natural template (see FIG. 8B).

Alternatively, the sequence architecture of a protein according to the invention comprising an ice-binding alpha helix and two stabilizing alpha helices has a “Helix-Loop-Helix-Loop-Helix” sequence architecture represented as H1-L1-H2-L2-H3, wherein H1 is the ice-binding helix featuring at least three copies of the ice-binding motif TXXXAXXXAXX, H2 and H3 are stabilizing alpha helices and L1 and L2 are loop sequences.

Alternatively, the sequence architecture of a protein according to the invention comprising an ice-binding alpha helix and two stabilizing alpha helices has a “Helix-Loop-Helix-Loop-Helix” sequence architecture represented as H1-L1-H2-L2-H3, wherein H3 is the ice-binding helix featuring at least three copies of the ice-binding motif TXXXAXXXAXX, H1 and H2 are stabilizing alpha helices and L1 and L2 are loop sequences.

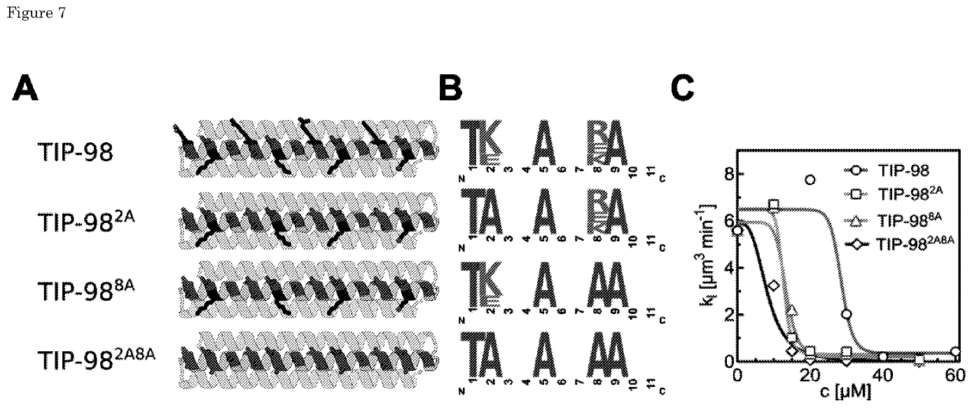

TABLE 1
Preferred sequences of a protein according to the
invention (as given by formula I). The * indicates
the four preferred alternatives for H2_{M}, all
providing good ice binding results as shown in
FIG. 7C, wherein TIP-98 corresponds to a helix-
loop-helix-loop-helix structure comprising the
sequence H2_{M}-1, TIP-98^{2A} corresponds to a
structure comprising H2_{M}-2, TIP-98^{8 A}corresponds
to a structure comprising H2_{M}-3 and TIP-98^{2A8A}
corresponds to a structure comprising H2_{M}-4.

| Element of the sequence | |
|---|---|
| architecture of an IBP | Amino acid sequence |
| H1_{N} | EEEAL |
| H1_{C} | EKLKKL |
| H2_{N} | DEATI and DAATI |
| H2_{C} | TEASAN and TAASAN |
| H3_{N} | SEEAL |
| H3_{C} | ERLDRN |
| L1 | GK |
| L2 | GV |
| H1_{M} | XXLXXXVXXA[L/E] |
| H2_{M}-1* | TXAXAXLXA[I/L]V |
| H2_{M}-2* | TAAXAXLXA[I/L]V |
| H2_{M}-3* | TXAXAXLAA[I/L]V |
| H2_{M}-4* | TAAXAXLAA[I/L]V |
| H3_{M} | XXLXXILXXA[L/E] |

For the protein visualized in FIG. 8A, the pair of capping sequences H2_{N} and H2_{C} together account for the fourth repetition of the TXXXAXXXAXX motif, next to the three uninterrupted central repetitions of the TXXXAXXXAXX motif represented by H2_{M}.

The total number of amino acid residues per helix in a protein according to the invention preferably is between 33 and 110 amino acid residues, more preferable between 44 and 88, more preferable between 44 and 55 amino acid residues, most preferably about 44 amino acid residues.

The total number of amino acid residues per protein according to the invention preferably is between 33 and 350 amino acid residues, more preferably between 100 and 334 amino acid residues, more preferably between 103 and 334 amino acid residues, most preferably about 136 amino acid residues. These numbers may vary depending on the presence of a C-terminal his-tag, such as GGSWHHHHHH (i.e. an additional 10 amino acid residues) and/or starting amino acid residue methionine, M (i.e. one additional amino acid residue).

A protein according to the invention may be modified. Said modification may be applied during or after synthesis of the protein. Said modification may include, for example, acetylation, phosphorylation, glycosylation and/or aminated. By selecting a particular host cell for expression of the protein, and/or by carrying out in vitro reactions, a person skilled in the art is able to modify an IBP according to the invention.

In particular good results in terms of IRI activity and thermal stability were obtained with proteins comprising a sequence having at least 80% sequence identity, preferably 90% sequence identity, most preferably 100% sequence identity with any one of SEQ NO: 5-8.

Without being bound to theory the high thermal stability of a protein according to the invention follows from well packed hydrophobic core and absence of hydrophobic residues on the surface of the protein. This means that surface residues are simultaneously designed to be high polar, making the proteins highly soluble and reducing in vivo aggregation, which is advantageous for expression.

### 4.4 Thermal Stability of an Ice-Binding Protein

A protein according to the invention is thermally stable at temperatures above 20° C., such as above 30° C., such as above 65° C., above 75° C., above 85° C. or even above 95° C. With thermal stability is meant that proteins can withstand these temperatures without unfolding and denaturing. A protein according to the invention is considered thermally stable if the structure of the alpha helix is remained at an elevated temperature such as above 20° C., above 30° C., above 65° C., above 75° C., above 85° C., or even above 95° C.

The advantage of a protein that is thermally stable, such as a protein according to the invention, is that the production of such protein is facilitated compared to a protein that is not thermally stable. In protein production processes, high temperatures are used for purification. Additionally, in the production process of a thermally stable protein, highly efficient lysis with combination of temperature and sheering forces can be used, leading to an increased product yield. Furthermore, high thermal stability correlates with high chemical stability meaning that a thermally stable protein, such as a protein according to the invention, is better protected against denaturing agents that may be present in some application environments. For example, environments comprising organic co-solvents such as DMSO used in cryopreservation, high salt concentrations or chaotropic agents or surfactants.

The thermal stability of a protein according to the invention can be characterized by investigating the protein's structure and in particular the conformation of the helix/helices at different temperatures. There are various methods known to a skilled person including, but not limited to, circular dichroism (CD), X-ray crystallography, electron crystallography and nuclear magnetic resonance spectroscopy (NMR) spectroscopy.

Circular dichroism (CD) spectroscopy is a form of light absorption spectroscopy that measures the difference in absorbance of right- and left-circularly polarized light (rather than the commonly used absorbance of isotropic light) by a protein according to the invention. It has been shown that CD spectra between approximately 260 and approximately 180 nm can be analyzed for the different secondary structural types: alpha helix, parallel and antiparallel beta sheet, turn, and other. For example, α-helical proteins may have negative bands at 222 nm and 208 nm of similar magnitude and a positive band at 193 nm.

X-ray crystallography uses X-ray to determine the position and arrangement of atoms in a crystal of a protein according to the invention. The most classical method of X-ray crystallography is single crystal X-ray diffraction, in which crystal atoms cause the incident X-ray beam to produce scattered beams. When the scattered beams land on the detector, these beams produce a speckle diffraction pattern. As the crystal is gradually rotated, the angle and intensity of these diffracted beams can be determined, and a three-dimensional image of the electron density within the crystal can be generated. Based on this electron density, information of the crystal of a protein such as the average position of atoms in the crystal, chemical bonds and crystal barriers can be determined. X-ray crystallography can be applied to confirm the structure of a protein including the presence of one or more helices, as well as the twisting of these helices.

Cryo-electron microscopy (Cryo-EM) includes three different methods: single particle analysis, electron tomography and electron crystallography. An essential feature of Cryo-EM is electron scattering, by which coherent electrons are used as a light source and a lens system converts the scattered signal into an image recorded on the detector. Signal processing is performed to obtain the three-dimensional structure of the sample.

Nuclear magnetic resonance (NMR) spectroscopy makes use of the fact that nuclei are charged, fast spinning elements. The gyromagnetic ratios of different atomic nuclei are different and therefore have different resonance frequencies. The movement of the nucleus is not isolated, it interacts with the surrounding atoms both intra- and inter-molecularly. Therefore, through NMR spectroscopy, structural information of a protein according to the invention can be obtained.

Based on the atomic structure as characterized by methods as listed herein above, such as CD, X-ray crystallography, electron crystallography and NMR spectroscopy, the helix twist (ω1) of an ice-binding helix can be calculated using software programs. For example, the helix twist (ω1) can be determined by using HELENAL to calculate the running average over 11 residues using the helenal_main function in MDAnalysis (Bansal et al., 2000. J Biomol Struct Dyn 17:811-819).

### 4.5 IRI Activity of an Ice-Binding Protein

Ice recrystallization is a thermodynamically driven process during which the ice grain boundary area per unit volume decreases. As this lowers the free energy of the system, it occurs spontaneously. There are three types of recrystallization processes: isomass, accretive, and migratory recrystallization. During isomass recrystallization, ice crystals change shape or internal structure, as irregular grain surfaces are rounded-off and ice crystal defects are reduced. During accretive recrystallization, two or more neighboring crystals merge into one. During migratory recrystallization, also known as Ostwald ripening, large crystals grow at the expense of small ones. The ice recrystallisation phenomenon is very complex and well described in literature, for example by Capicciotti et al. (2013, book: Recent Developments in the Study of Recrystallization, chapter: Ice Recrystallization Inhibitors: From Biological Antifreezes to Small Molecules, editor: P Wilson, DOI: 10.5772/54992).

IRI activity of a protein can be determined by investigating the degree of ice recrystallisation using various methods known to a person skilled in the art. These methods include splat cooling assay (SCA) and sucrose sandwich assay (SSA), providing visual comparisons of ice crystal structures using a microscope. Both of these assays probe the rate and extent of ice recrystallization in thin wafers of ice. Splat assays are typically performed in the presence of >2 mM NaCl or 1-100 mM phosphate-buffered saline (PBS) buffer and sandwich assays in the presence of 18-45% sucrose. In a splat assay quantification of IRI efficacy is based on measurements of the (time-evolution of the) mean largest grain size (MLGS). In a sandwich assay the inhibitory concentration Cᵢ, is taken as a quantitative measure for IRI activity. It demarcates the boundary between a high recrystallization rate ka at low IBP concentration C_{IBP} and a low ka at high C_{IBP}.

IRI activity can also be determined by X-ray powder diffraction (XRD) as extensively described by Fayter et al. (Fayter et al., 2020. Analyst 145:3666-3677). Using XRD, 3D information can be obtained.

### 4.6 Producing an Ice Binding Protein

The invention furthermore relates to an in vitro method of producing a protein according to the invention.

A protein according to the invention can be obtained by expression in a suitable expression system. Commonly used expression systems for heterologous protein production include host cells such as Escherichia coli, Bacillus spp., baculovirus, yeast, fungi, filamentous fungi or yeasts such as Saccharomyces cerevisiae and Pichia pastoris, mammalian cells such as Chinese Hamster Ovary cells (CHO), human embryonic kidney (HEK) cells and PER.C6® cells (Thermo Fisher Scientific, MA, USA), and plants. Said host cell may be a thermophilic cell such as Thermus aquaticus, Sulfolobus solfataricus and S. acidocaldarius.

A protein according to the invention preferably is produced using prokaryotic cells such as E. coli. Said protein is preferably produced by expression cloning of the proteins in a prokaryotic cell of interest, preferably E. coli. For this, an expression vector is preferably produced by recombinant technologies, including the use of polymerases, restriction enzymes, and ligases, as is known to a skilled person. Alternatively, said expression vector is provided by artificial gene synthesis, for example by synthesis of partially or completely overlapping oligonucleotides, or by a combination of organic chemistry and recombinant technologies, as is known to the skilled person.

Said expression vector may be codon-optimised to enhance expression of the protein of the invention in a host cell of interest, such as E. coli. Further optimization may include the removal of cryptic splice sites, removal of cryptic polyA tails and/or removal of sequences that may lead to unfavorable folding of the mRNA. In addition, the expression vector may encode a protein export signal for secretion of the protein of the invention out of the cell into the periplasm of prokaryotes, allowing efficient purification of the protein of the invention.

Methods for purification of the protein of the invention are known in the art and are generally based on chromatography such as affinity chromatography and ion exchange chromatography, to remove contaminants. In addition to contaminants, it may also be necessary to remove undesirable derivatives of the product itself such as degradation products and aggregates. Suitable purification process steps are provided in Berthold and Walter, 1994 (Berthold and Walter, 1994. Biologicals 22:135-150).

As an alternative, or in addition, a recombinant protein according to the invention may be tagged with one or more specific tags by genetic engineering to allow attachment of the protein to a bead or column that is specific to the tag and therefore be isolated from impurities. The purified protein is then exchanged from the affinity bead or column with a decoupling reagent. The method has been routinely applied for purifying recombinant protein. Conventional tags for proteins, such as histidine tag, are used with an affinity bead or column that specifically captures the tag (e.g., a Ni-IDA column for the histidine tag) to isolate the protein from other impurities. The protein is then exchanged from the bead or column using a decoupling reagent according to the specific tag (e.g., imidazole for histidine tag). This method is more specific, when compared with traditional purification methods.

Suitable tags include c-myc domain, hemagglutinin tag maltose-binding protein, glutathione-S-transferase, FLAG tag peptide, biotin acceptor peptide, streptavidin-binding peptide and calmodulin-binding peptide, as presented in Chatterjee, 2006 (Chatterjee, 2006. Cur Opin Biotech 17, 353-358). Methods for employing these tags are known in the art and may be used for purifying a protein according to the invention.

Methods for expression of proteins in E. coli are known in the art and can be used for expression and optionally purification of a protein of the invention.

In a preferred method, a protein according to the invention is expressed in E. coli from a synthetic DNA encoding the protein according to the invention. Said DNA is cloned into an expression vector and the protein is purified by HisTag immobilized-metal affinity chromatography.

In an embodiment, a protein according to the invention may be obtained by peptide synthesis of the helices, such as at least two of H1, H2 and H3, preferably at least one ice binding helix (H2) and one stabilizing helix (H1 or H3), or at least one ice binding helix (H2) and two stabilizing helices (H1 and H3), individually. Afterwards, the individual helices may be mixed and at least a fraction of the helices will form a heterodimer of H2 and H1 or of H2 and H3, or a heterotrimer of H1, H2 and H3. In the mixing process, also other trimers may be formed such as homotrimers of H1, H2 or H3 or heterotrimers having a double copy of one of the helixes. A heterotrimer of H1, H2 and H3 will result in a protein being more stable than the other trimer forms.

In another embodiment, a protein according to the invention may be obtained by producing a heterotrimer of H1, H2 and H3 by using a bicistronic or tricistronic construct, meaning that two or three helices are expressed by one vector respectively.

### 4.7 Use of an Ice Binding Protein

A protein according to the invention is able to reduce or prevent ice recrystallisation as well as the formation of (sharp) ice crystals during freezing and thawing, making it especially useful as a cryopreservation agent.

The invention provides a composition comprising an effective amount of a protein according to the invention. Said composition may furthermore comprise water, DMSO, glycerol, trehalose, fetal calf serum (FCS), cell culture medium, a buffer e.g. PBS, an antibiotic, an anti-coagulant, an anti-oxidant and/or a pH indicator. Said composition can be used as cryopreserving composition and is suitable for the preservation of biological material and food products. In the case of the preservation of biological material, the composition is a physiologically acceptable composition.

A protein according to the invention or an composition comprising said protein can be used in a method for cryopreservation of an aqueous mixture. In such method, the aqueous mixture or part of the aqueous mixture is brought into contact with the protein, or with a composition comprising said protein.

A key problem in cryopreservation of biological materials, such as cells or tissues and/or organs, is that freezing and thawing often results in damage caused by sharp ice crystals that puncture the cell membrane. A protein according to the invention was shown to prevent ice recrystallization and the formation of (sharp) ice crystals growth during freezing and thawing and therefore is especially beneficial for use in a method for cryopreservation of biological materials.

A protein or composition according to the invention may be added to a food product by contacting the entire product with said protein or composition, or alternatively may be applied to only the surface or only a part of the food product. A protein or composition according to the invention may be added during the preparation of the food product, prior to freezing, during freezing, and/or after freezing of the product.

Many frozen food products suffer from growth of ice during storage which can adversely affect the quality of the product e.g. in terms of texture and flavour. Ice crystal growth is undesirable during food product freezing since it can induce morphological and mechanical changes and/or cellular damage. For example, frozen ice cream often comprises large crystals resulting in a grainy texture. Another example is a frozen fruit or meat product which tends to lose significant volumes of water when it is frozen and defrosted afterwards, changing its texture.

A protein according to the invention has IRI activity, meaning that it can minimise or even prevented ice crystal growth. Therefore, a protein according to the invention or a composition comprising said protein can be used in a method for cryopreservation of a food product. An advantage of such method is that the quality of the food product is maintained since crystal growth is prevented or minimised, when compared to a food product that was not contacted with said protein or composition. Additionally, such method also increases the shelf-life of frozen food products.

A protein according to the invention or a composition comprising said protein can be used for de-icing materials such as aircraft wings, drones, air conditioners, refrigerators, freezers, electricity cables, window shields or structures of wind turbines such as blades. The use of a protein according to the invention for such materials can prevent, inhibit or delay the formation of ice on said materials.

Furthermore, a protein according to the invention or a composition comprising said protein can be used as a gas hydrate inhibitor. Gas hydrates are ice-like clathrate structures composed of water cages surrounding trapped gas molecules, which, depending on the gas, can form at temperatures above 0° C. and at modest pressures (0.5 to several MPa). Although gas hydrate deposits are a potential energy source, the unscheduled formation of gas hydrates is a major problem for the petroleum industry, since they can cause blockages at well heads and inside pipelines, with potentially disastrous consequences. It was shown that IBPs can inhibit gas hydrate propagation, notwithstanding the distinct differences in the crystal structures of hydrates and ice.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

Sequences
All sequences provided herein are from N-terminus
to C-terminus.
SEQ NO: 1 (sequence of the [TXXXAXXXAXX]₃ repeat
of the ice-binding alpha helix of TIP-98TIP-98)
TKAKAKLRAIVTKAEADLRALVTKAEAKLKAIV
SEQ NO: 2 (sequence of the [TXXXAXXXAXX]₃ repeat
of the ice-binding alpha helix of TIP-98^{2A})
TAAKAKLRAIVTAAEADLRALVTAAEAKLKAIV
SEQ NO: 3 (sequence of the [TXXXAXXXAXX]₃ repeat
of the ice-binding alpha helix of TIP-98^{8A})
TKAKAKLAAIVTKAEADLAALVTKAEAKLAAIV
SEQ NO: 4 (sequence of the [TXXXAXXXAXX]₃ repeat
of the ice-binding alpha helix of TIP-98^{2A8A})
TAAKAKLAAIVTAAEADLAALVTAAEAKLAAIV
SEQ NO: 5 (Sequence of TIP-98)
MEEEALKKLKDTVKEALKRLKELVDRALKKLKETVKRAEEKLKKLGKDEA
TITKAKAKLRAIVTKAEADLRALVTKAEAKLKAIVTEASANGVSEEALER
LERILREALKRLKKILKEALERLKKILKTAEERLDRNSGGWHHHHHH
SEQ NO: 6 (Sequence of TIP-98^{2A})
MEEEALKKLKDTVKEALKRLKELVDRALKKLKETVKRAEEKLKKLGKDEA
TITAAKAKLRAIVTAAEADLRALVTAAEAKLKAIVTAASANGVSEEALER
LERILREALKRLKKILKEALERLKKILKTAEERLDRNSSGWHHHHHH
SEQ NO: 7 (Sequence of TIP-98^{8A})
MEEEALKKLKDTVKEALKRLKELVDRALKKLKETVKRAEEKLKKLGKDAA
TITKAKAKLAAIVTKAEADLAALVTKAEAKLAAIVTEASANGVSEEALER
LERILREALKRLKKILKEALERLKKILKTAEERLDRNSSGWHHHHHH
SEQ NO: 8 (Sequence of TIP-98^{2A8A})
MEEEALKKLKDTVKEALKRLKELVDRALKKLKETVKRAEEKLKKLGKDAA
TITAAKAKLAAIVTAAEADLAALVTAAEAKLAAIVTAASANGVSEEALER
LERILREALKRLKKILKEALERLKKILKTAEERLDRNSSGWHHHHHH
SEQ NO: 9 (Sequence of TIP-99ₐ)
MEEEAKKKIDDLLTKARREVKKAIKTAREVAKRASKKIEELERRNEDKEA
AATKMEAILRAVKTTMKALIEALRTQMKAAAKAMKTIVKAEPESEELKKK
VEDAIKDMRRLVEEAIREMEKLARELEKQAREAQKRTSGGWHHHHHH

### 5 EXAMPLES

### Materials and Methods

### Synthetic Gene Construction

Gene fragments encoding Twist constrained Ice binding Proteins (TIP) were codon optimized using Codon Harmony (1.0.0) and obtained as synthetic DNA fragments from Twist Bioscience. The gene fragments were cloned into a modified pET-24(+) expression vector using standard restriction cloning with BamHI and XhoI restriction endonucleases. The cloned plasmid DNA inserts were sequence verified using Sanger sequencing and transformed into T7-Express Escherichia coli (NEB) via heat-shock.

### Bacterial Protein Expression and Purification

A 25 mL terrific broth culture supplemented with 50 mg/L kanamycin antibiotic was inoculated. The starter culture is grown overnight at 37° C. in a shaker and used to inoculate 1 L of Miller's LB Broth Base (10 g tryptone, 10 g NaCl and 5 g yeast extract, Invitrogen) supplemented with 50 mg/L kanamycin. The culture was incubated shaking until 0.6<OD₆₀₀<0.8 at 37° C. in a 2 L baffled Erlenmeyer. Protein expression was induced by 1 mM isopropyl B-D-thiogalactoside (IPTG) and expression was continued at 18° C. overnight. The cell broth was centrifuged at 6,000×g and the cell pellet was resuspended in ice-cold 30 mL lysis-wash buffer (50 mM Tris-HCl pH 8.0, 300 mM NaCl, 30 mM imidazole) supplemented with 1 mM phenylmethylsulfyl fluoride (PMSF) serine protease inhibitor and a pinch of DNAseI. Cells were then lysed by sonication on ice for 7 min. with a 2 s on-off duty cycle at 85% amplitude using a Qsonica Q125 with a CL-18 probe. The lysate was centrifuged at 30,000×g for 30 min. at 4° C. and the clarified supernatant was applied two times on lysis-wash buffer equilibrated Ni-NTA resin with a column volume (CV) of ˜2 mL. The resin was washed with 25 CVs lysis-wash buffer and eluted with 3 CVs elution buffer (25 mM Tris-HCl pH 8.0, 300 mM NaCl, 300 mM imidazole). Eluted protein was dialyzed to PBS+ (10 mM phosphate pH 7.4+300 mM NaCl) and further purified by size-exclusion chromatography on a Superdex 75 10/300 (GE Healthcare) in PBS+ on an Agilent infinity II. Protein purity was analyzed by SDS-PAGE and purified protein was concentrated to ˜20 mg/mL and stored at 4° C.

### Circular Dichroism

TIP proteins were diluted to 0.15 mg/mL in PBS+ in a quartz cuvette with a 1 mm pathlength. On a JASCO J-715 (JASCO Corporation) spectral scans were averaged over 20 measurements with scan rate of 50 nm/min and a response time of 2 s. A spectral scan was performed at 20° C., followed by thermal ramp to 95° C. at 220 nm with rate of 1° C./min. After reaching 95° C. the temperature was reversed to 20° C. (20° C. rev), and another spectral scan was performed. Data where the high tension was above 600V is not shown.

### Ice Recrystallization Inhibition

Samples were prepared by dilution of the protein to the indicated concentration in 20 wt % sucrose in PBS+. Subsequently, a 2 μL sample was applied on a 22×22 μm coverslip and a second coverslip was lowered on top of the drop so that the sample was sandwiched between the coverslips and transferred to a Nikon ECLIPSE Ci-Pol Optical Microscope equipped with a Nikon L Plan 20× (NA 0.45) objective and the Linkam LTS420 stage. This stage was controlled by the Linksys32 software. To measure the ice-recrystallization rates, the sample was first completely frozen by reducing the temperature to −40° C. with 20° C./min. After freezing the temperature was gradually increased to −10° C. with 10° C./min and then further to −7° C. with 1° C./min upon which individual crystals could be observed and the sample was stabilized. Recrystallization was monitored by obtaining an image each minute. IRI rates were then analyzed using ImageJ and Matlab. In brief, the 8-bit images of the ice-crystals were subjected to the bandpass filter, enhance contrast and subtract background function of imageJ. Subsequently, the bright signal at the edges and then the individual crystals were isolated by the autoThreshold and Convert to Mask function. Analyze Particles was used to obtain the area of each crystal. This data was imported into Matlab upon which the radius was calculated in order to determine the corresponding spherical volume of each crystal. Recrystallization growth rates were determined by applying a linear fit to the resulting ice-volume as a function of time traces.

### Ice Shaping Assay

To monitor ice-shaping in the presence of the various TIPs, the sample was prepared as for the IRI assays but now the sample was stabilized at −4.3° C., so that even fewer crystals were observed in the field of view. After stabilization, the samples were supercooled with 0.2° C. per minute which forced the crystals to shape in presence of the TIP proteins or type-I AFP purified from winter flounder (wfAFP) (Tas et al., 2022. bioRxiv 2022.04.05.487137). Samples were monitored with 1 second intervals using a Nikon 50×ELWD objective. Stills that were used in the figures were taken after 1 minute of supercooling at −4.5° C.

### Crystallization

Crystallization samples were prepared by concentrating TIP-99a protein to 20 mg/mL in PBS+. All crystallization experiments were conducted using the sitting drop vapor diffusion method. Crystallization trials were set up in 200 nL drops using the 96-well plate format at 20° C. Crystallization plates were set up using a Mosquito from SPT Labtech, then imaged using UVEX microscopes and UVEX PS-600 from JAN Scientific. Diffraction quality crystals formed in 0.02M 1,6-hexanediol, 0.02M 1-butanol, 0.02M 1,2-propanediol, 0.02M 2-propanol, 0.02M 2-propanol, 0.02M 1,4-butanediol, 0.02M 1,3-propanediol, 0.0466M pH 8.5 Tris (base), 0.0534M pH 8.5 Bicine, 20% v/v PEG 500 MME, and 10% w/v PEG 20,000.

X-ray intensities and data reduction were evaluated and integrated using XDS (Glusker et al., 1993. Acta Cryst 49:1) and merged/scaled using Pointless/Aimless in the CCP4 program suite (Winn et al., 2011. Acta Cryst 67:235-242). Structure determination and refinement starting phases were obtained by molecular replacement using Phaser (McCoy et al., 2007. J Appl Crystallogr 40:658-674) using the designed model for the structures. Following molecular replacement, the models were improved using phenix.autobuild (Adams et al., 2010. Acta Cryst 66:213-221); efforts were made to reduce model bias by setting rebuild-in-place to false, and using simulated annealing and prime-and-switch phasing. Structures were refined in Phenix (McCoy et al., 2007. J Appl Crystallogr 40:658-674). Model building was performed using COOT (Emsley & Cowtan, 2004. Acta Cryst 60:2126-2132). The final model was evaluated using MolProbity (Williams et al., 2018. Protein Sci 27:293-315).

### EXAMPLE 1: DE NOVO DESIGN OF HYPER-STABLE IBPS

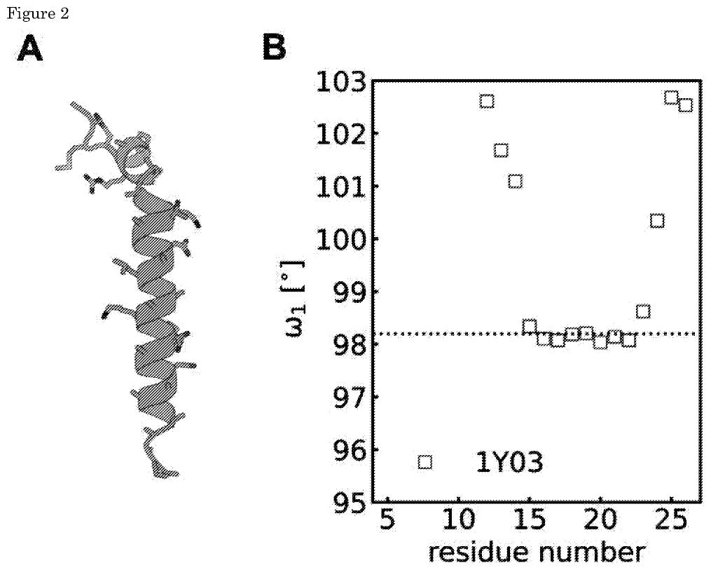

Natural helical IBPs, such as the type I sculpin AFP and winter flounder AFP (wfAFP), contain at least 2 or at least 3 repeats of an 11 residue consensus sequence TXXXAXXXAXX (where X can be any amino acid) respectively. From two solved crystal structures of helical IBPs (protein data bank (pdb) id: 1WFA, i.e. wfAFP, FIG. 1A; and pdb: 1Y03, i.e. type I sculpin AFP, FIG. 2A) it was observed that the helical backbone of the ice binding consensus sequences adopts a characteristic twist of 98.2 degrees per residue, such that the threonine residues (T) are all facing in the same direction (FIG. 1A-B, FIG. 2A-B). However, a relaxed straight helix tends to adopt a helical twist of approximately 100-101 degrees per residue (FIG. 1C). From these observations it was postulated that the design of an artificial de novo helical IBP would require a straight helix with the minimal ice binding consensus sequence TXXXAXXXAXX that is under-twisted into a twisting of precisely 98.2 degrees per residue (FIG. 1D).

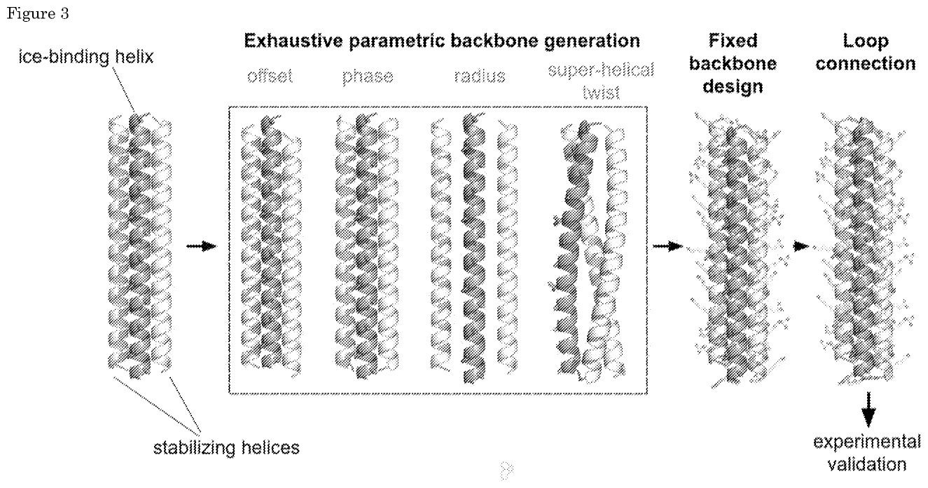

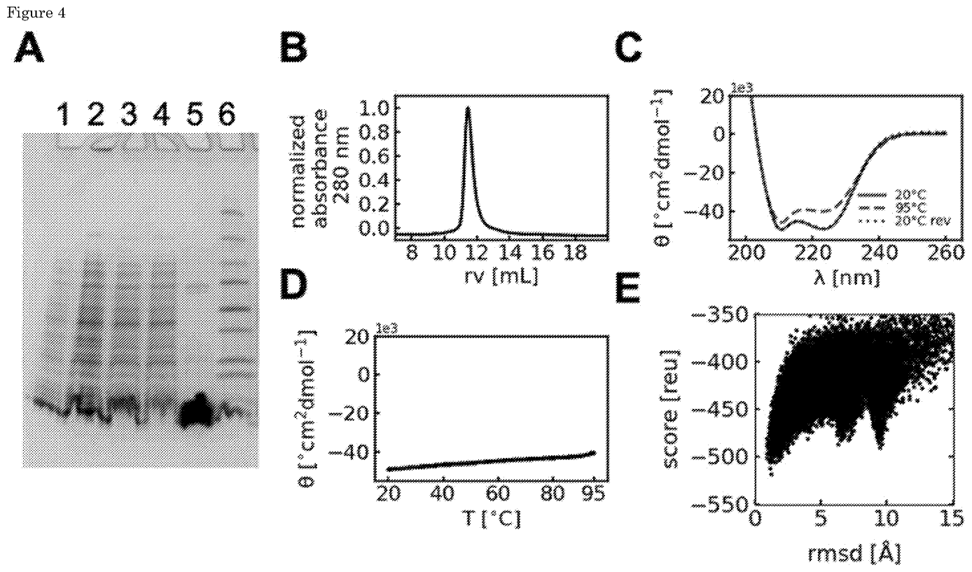

Next, proteins were designed in which an under-twisted helical twist of precisely 98.2 degrees per residue of an ice-binding helix was forced by using two ‘stabilizing’ helices in a bundle (FIG. 3). To achieve this, parameterized Watson-Crick helix equations were used to generate a large library of helix bundles in a 3-fold cyclic symmetry group (C3) with a helical twist of 98.2 degrees per residues. For each of the three helices in the bundle, different helix parameters were sampled independently such as the phase, offset, radii and super-helical curvature (FIG. 3). In this way, more than 70,000 unique backbone designs were combinatorically generated (FIG. 3). Next, the ice binding consensus residues on the ice-binding helix were fixed and each backbone was further populated with amino acid rotamers using the PackRotamersMover in the Rosetta protein design software (fixed backbone design) (FIG. 3). In the design procedure types of amino acid residues were left free, except for the threonines and alanines in the 11 residue consensus sequence TXXXAXXXAXX on the central ice-binding helix. After this, the three helices were connected into a bundle. The Rosetta Remodel package was used to design connecting residue loops of 2-3 residues long (FIG. 3). Designs were filtered based on the potential energy of the structure and root-mean square displacement relative to the models relaxed without any backbone constraints. Lastly, for the selected designs ab initio protein folding simulations starting from the extended chain were performed using the Rosetta AbinitoRelax module to generate protein folding energy landscapes (FIG. 4E). Designs that showed a clear ‘funnel’ behavior-indicative of a single lowest energy state-were selected and synthetic DNA encoding the protein was obtained, cloned into an expression vector and the protein was purified by HisTag immobilized-metal affinity chromatography from an E. coli culture and further characterized experimentally.

Out of the 5 designs tested, 1 design “TIP-98” had very high expression in E. coli (FIG. 4A), was monomeric by size-exclusion chromatography (SEC, FIG. 4B) and showed a clear alpha-helical signal in circular dichroism (CD, FIG. 4C-D). Moreover, the design retained its alpha helical structure at temperatures >95° C. (FIG. 4C-D).

### EXAMPLE 2: IBPS WITH IMPROVED IRI ACTIVITY

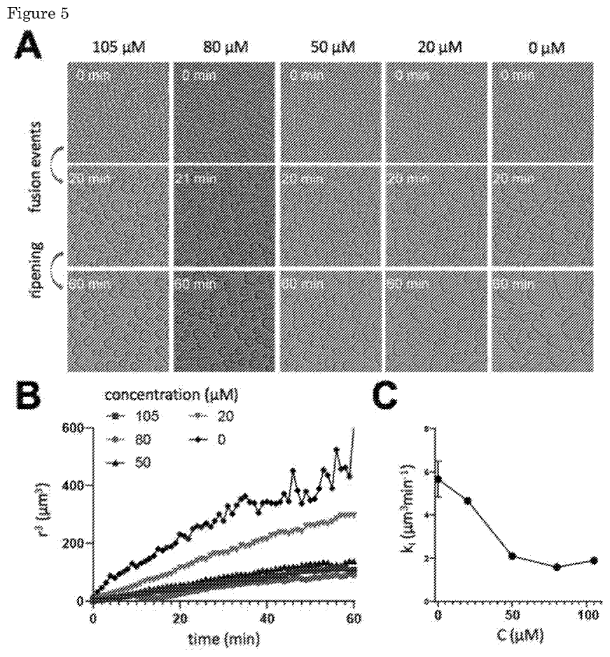

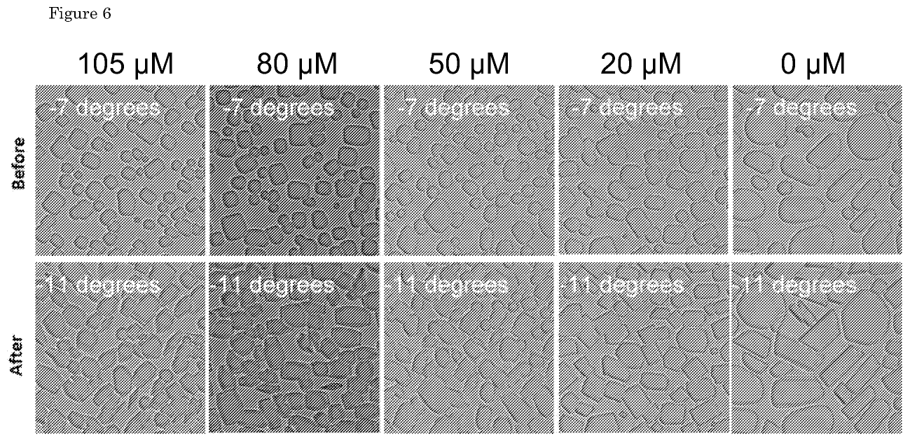

Ice recrystallization inhibition (IRI) activity assays of TIP-98 shows that ice crystal growth is significantly slowed down between 20 μM and 50 μM (FIG. 5A,B,C), which is on par with existing natural IBPs on which these designs are based. At concentrations of 20 UM and higher, the TIP-98 design shapes ice into mostly blunt-end crystals, with a morphology distinctly different from that of ice crystals in the absence of TIP-98 (FIG. 6). This suggests that TIP-98 adsorbs onto specific planes of the ice crystals, which blocks further growth in this direction.

Several mutants of the TIP-98 have been designed which have (enhanced) IRI activity (FIG. 7). It is hypothesized that matching better the ice binding residues from the natural wfAFP will increase activity. Therefore, high entropy, charged and polar side chains were mutated near the ice-binding residues to alanines (FIG. 7A-B). These mutations outperformed the TIP-98 and halt ice growth completely at 20 μM (FIG. 7C). The sequences of TIP-98 and mutants are shown in Table 2.

### EXAMPLE 3: 3D STRUCTURES OF AIBPS

In FIG. 8 a computationally obtained structure for a helical bundle TIP with a helix-loop-helix-loop-helix structure (indicated by H1-L1-H2-L2-H3) with a central ice binding helix H2 where the minimal ice-binding consensus sequence is shown. The helices consists of 4 repeats (rep), where the edges are defined as two halve repeats (0.5 rep) and the middle contains 3 full repeats of the 11-mer sequence TXXXAXXXAXX. Computationally obtained structures demonstrate tight core packing of hydrophobic amino acid residues in 11-mer sequences (FIG. 8B). The spatial arrangement of the active ice-binding amino acid residues is maintained in the same spatial arrangement as in the natural template, in this case the wfAFP.

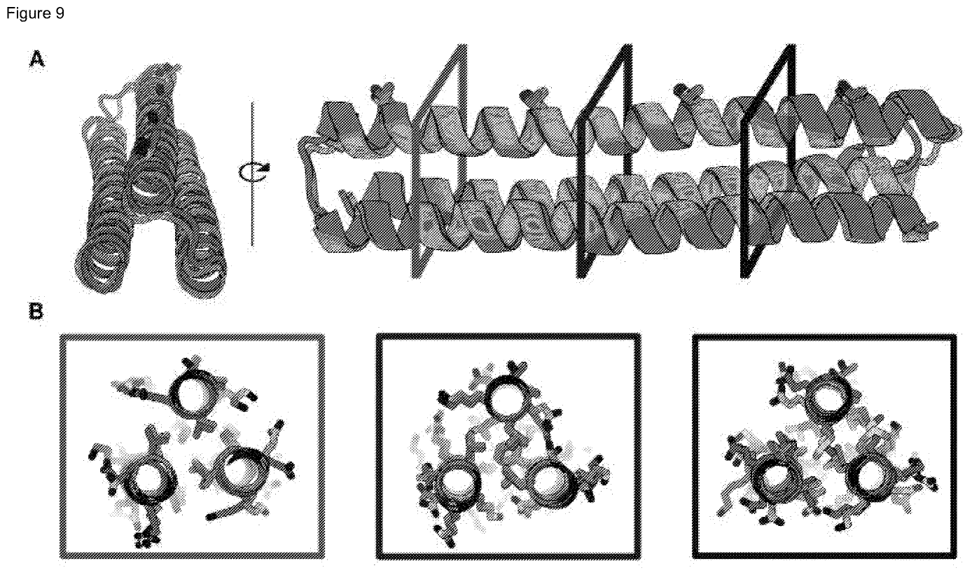

As a comparative example, the crystal structure of a protein having a helical twisting of the ice-binding helix and the ice-binding residues of 99.2° per residue (i.e. TIP-99a) was determined. A 3D structure of TIP-99a shows a helical twisting of 99.2° per residue, resulting in a rotamer packing in the crystal structure that shows that the threonine residues are not all facing to the same direction (FIG. 9 A-B). This is in contrast to what was observed for the TIP-98 designs, i.e. proteins with a twist of less than 99° per residue, such as 98.2 degrees per residue.

TABLE 2
overview of designs with alanine mutations that
show higher IRI activity compared to the TIP-98.

| Design | |
|---|---|
| name | Protein sequence (N-C terminus) |
| TIP-98 | MEEEALKKLKDTVKEALKRLKELVDRALKKLKETVKR |
| | AEEKLKKLGKDEATITKAKAKLRAIVTKAEADLRALVT |
| | KAEAKLKAIVTEASANGVSEEALERLERILREALKRLK |
| | KILKEALERLKKILKTAEERLDRNSGGWHHHHHH |
| TIP- | MEEEALKKLKDTVKEALKRLKELVDRALKKLKETVKR |
| 98^{2A} | AEEKLKKLGKDEATITAAKAKLRAIVTAAEADLRALVT |
| | AAEAKLKAIVTAASANGVSEEALERLERILREALKRLK |
| | KILKEALERLKKILKTAEERLDRNSSGWHHHHHH |
| TIP- | MEEEALKKLKDTVKEALKRLKELVDRALKKLKETVKR |
| 98^{8A} | AEEKLKKLGKDAATITKAKAKLAAIVTKAEADLAALVT |
| | KAEAKLAAIVTEASANGVSEEALERLERILREALKRLK |
| | KILKEALERLKKILKTAEERLDRNSSGWHHHHHH |
| TIP- | MEEEALKKLKDTVKEALKRLKELVDRALKKLKETVKR |
| 98^{2A8A} | AEEKLKKLGKDAATITAAKAKLAAIVTAAEADLAALVT |
| | AAEAKLAAIVTAASANGVSEEALERLERILREALKRLK |
| | KILKEALERLKKILKTAEERLDRNSSGWHHHHHH |

1. A protein comprising at least one ice-binding alpha helix and one or more stabilizing alpha helices, wherein the ice-binding helix has a twist of less than 100 degrees per residue.

2. The protein according to claim 1, wherein the at least one ice-binding alpha helix comprises at least two copies of sequence TXXXAXXXAXX, wherein T represents a threonine residue, A least three copies of sequence TXXX/KX represents an alanine residue and X represents any amino acid residue.

3. The protein according to claim 1, wherein the at least one ice-binding alpha helix comprises at least two copies of sequence TXAXAXLXAX[I/L]V, wherein T represents a threonine residue, A represents an alanine residue, I represents an isoleucine residue, L represents an leucine residue, V represents an valine residue and X represents any amino acid.

4. The protein according to claim 1, wherein the one or more stabilizing alpha helices are linked to the at least one ice-binding alpha helix.

5. The protein according to claim 1, wherein the protein is thermally stable such that the structure of the alpha helix is remained at temperatures above 20° C.

6. The protein according to claim 1, wherein the total number of amino acid residues of the protein is between 33 and 350 amino acid residues.

7. The protein according to claim 1, wherein the protein comprises a sequence having at least 80% sequence identity with any one of SEQ NO: 1-8.

8. A composition comprising an effective amount of the protein according to claim 1 and one or more other components selected from water, DMSO, glycerol, trehalose, fetal calf serum, cell culture medium, a buffer, an antibiotic, an anti-coagulant, an anti-oxidant, and a pH indicator.

9. (canceled)

10. (canceled)

11. A method of stabilizing an ice-binding alpha helix, comprising the steps of:
(a) providing an ice-binding alpha helix as described in claim 1;
(b) linking one or more alpha helices to the ice-binding alpha helix of the protein;
thereby stabilizing the ice-binding alpha helix.

12. A method for cryopreserving an aqueous mixture, comprising contacting the aqueous mixture with the protein according to claim 1.

13. The method according to claim 12, wherein the aqueous mixture is a biological material, a tissue, an organ, or part thereof.

14. The method according to claim 12, wherein the aqueous mixture is a food product.

15. A method for producing the protein according to claim 1, comprising the steps of:
(a) providing an expression vector comprising a nucleic acid encoding the protein in a host cell;
(b) expressing the protein; and
(c) optionally purifying the protein from the host cell.

16. The method according to claim 15, wherein the host cell is a bacterium.

17. The protein according to claim 1, wherein the ice-binding helix has a twist of less than 99 degrees per residue.

18. The protein according to claim 1, wherein the ice-binding helix has a twist of about 98.2 degrees per residue.

19. The protein according to claim 2, wherein the at least one ice-binding alpha helix comprises at least three copies of sequence TXXXAXXXAXX, wherein T represents a threonine residue, A represents an alanine residue, and X represents any amino acid residue.

20. The protein according to claim 3, wherein the at least one ice-binding alpha helix comprises at least three copies of sequence TXAXAXLXAX[I/L]V, wherein T represents a threonine residue, A represents an alanine residue, I represents an isoleucine residue, L represents an leucine residue, V represents an valine residue, and X represents any amino acid.

21. The protein according to claim 5, wherein the protein is thermally stable such that the structure of the alpha helix remains at temperatures above 30° C.

22. The protein according to claim 21, wherein the protein is thermally stable such that the structure of the alpha helix remains at temperatures above 65° C.

23. The protein according to claim 22, wherein the protein is thermally stable such that the structure of the alpha helix remains at temperatures above 75° C.

24. The protein according to claim 23, wherein the protein is thermally stable such that the structure of the alpha helix remains at temperatures above 85° C.

25. The protein according to claim 24, wherein the protein is thermally stable such that the structure of the alpha helix remains at temperatures above 95° C.

26. The protein according to claim 6, wherein the total number of amino acid residues of the protein is between 100 and 200 amino acid residues.

27. The method according to claim 14, wherein the food product is an ice cream, meat, a fruit or a vegetable.

28. The method according to claim 16, wherein the host cell is Escherichia coli.

29. A method to prevent or retard the formation of gas hydrates, comprising applying the protein according to claim 1.

30. A method to de-ice a material, comprising applying the protein according to claim 1 in a coating to the material.

31. The method of claim 30, wherein the material is an aircraft wing, drone, air conditioner, refrigerator, freezer, electricity cable, window shield or structure of wind turbine.


SEQUENCE LISTING (32 sequences)

SEQ ID NO: 1 - protein, 33 aa, synthetic construct
TKAKAKLRAIVTKAEADLRALVTKAEAKLKAIV

SEQ ID NO: 2 - protein, 33 aa, synthetic construct
TAAKAKLRAIVTAAEADLRALVTAAEAKLKAIV

SEQ ID NO: 3 - protein, 33 aa, synthetic construct
TKAKAKLAAIVTKAEADLAALVTKAEAKLAAIV

SEQ ID NO: 4 - protein, 33 aa, synthetic construct
TAAKAKLAAIVTAAEADLAALVTAAEAKLAAIV

SEQ ID NO: 5 - protein, 147 aa, synthetic construct
MEEEALKKLKDTVKEALKRLKELVDRALKKLKETVKRAEEKLKKLGKDEATITKAKAKLRAIVTKAEADLRALVTKAEAKLKAIVTEASANGVSEEALERLERILREALKRLKKILKEALERLKKILKTAEERLDRNSGGWHHHHHH

SEQ ID NO: 6 - protein, 147 aa, synthetic construct
MEEEALKKLKDTVKEALKRLKELVDRALKKLKETVKRAEEKLKKLGKDEATITAAKAKLRAIVTAAEADLRALVTAAEAKLKAIVTAASANGVSEEALERLERILREALKRLKKILKEALERLKKILKTAEERLDRNSSGWHHHHHH

SEQ ID NO: 7 - protein, 147 aa, synthetic construct
MEEEALKKLKDTVKEALKRLKELVDRALKKLKETVKRAEEKLKKLGKDAATITKAKAKLAAIVTKAEADLAALVTKAEAKLAAIVTEASANGVSEEALERLERILREALKRLKKILKEALERLKKILKTAEERLDRNSSGWHHHHHH

SEQ ID NO: 8 - protein, 147 aa, synthetic construct
MEEEALKKLKDTVKEALKRLKELVDRALKKLKETVKRAEEKLKKLGKDAATITAAKAKLAAIVTAAEADLAALVTAAEAKLAAIVTAASANGVSEEALERLERILREALKRLKKILKEALERLKKILKTAEERLDRNSSGWHHHHHH

SEQ ID NO: 9 - protein, 147 aa, synthetic construct
MEEEAKKKIDDLLTKARREVKKAIKTAREVAKRASKKIEELERRNEDKEAAATKMEAILRAVKTTMKALIEALRTQMKAAAKAMKTIVKAEPESEELKKKVEDAIKDMRRLVEEAIREMEKLARELEKQAREAQKRTSGGWHHHHHH

SEQ ID NO: 10 - protein, 5 aa, synthetic construct
EEEAL

SEQ ID NO: 11 - protein, 6 aa, synthetic construct
EKLKKL

SEQ ID NO: 12 - protein, 5 aa, synthetic construct
DEATI

SEQ ID NO: 13 - protein, 5 aa, synthetic construct
DAATI

SEQ ID NO: 14 - protein, 6 aa, synthetic construct
TEASAN

SEQ ID NO: 15 - protein, 6 aa, synthetic construct
TAASAN

SEQ ID NO: 16 - protein, 5 aa, synthetic construct
SEEAL

SEQ ID NO: 17 - protein, 6 aa, synthetic construct
ERLDRN

SEQ ID NO: 18 - protein, 11 aa, synthetic construct
  VARIANT 11: note = L or E
XXLXXXVXXAL

SEQ ID NO: 19 - protein, 11 aa, synthetic construct
  VARIANT 10: note = I or L
TXAXAXLXAIV

SEQ ID NO: 20 - protein, 11 aa, synthetic construct
  VARIANT 10: note = I or L
TAAXAXLXAIV

SEQ ID NO: 21 - protein, 11 aa, synthetic construct
  VARIANT 10: note = I or L
TXAXAXLAAIV

SEQ ID NO: 22 - protein, 11 aa, synthetic construct
  VARIANT 10: note = I or L
TAAXAXLAAIV

SEQ ID NO: 23 - protein, 11 aa, synthetic construct
  VARIANT 11: note = L or E
XXLXXILXXAL

SEQ ID NO: 24 - protein, 12 aa, synthetic construct
  VARIANT 11: note = I or L
TXAXAXLXAXIV

SEQ ID NO: 25 - protein, 40 aa, synthetic construct
TAAAAAAAAATAAAAAAAAATAAAAAAAAATAAAAAAAAA

SEQ ID NO: 26 - protein, 6 aa, synthetic construct
  VARIANT 4: note = D, E, H, K, N, Q, R or S
  VARIANT 6: note = D, E, H, K, N, Q, R or S
TAAXAX

SEQ ID NO: 27 - protein, 5 aa, synthetic construct
  VARIANT 1: note = D, E, H, K, N, Q, R or S
XAATI

SEQ ID NO: 28 - protein, 11 aa, synthetic construct
  VARIANT 11: note = L or E
XXLXXXVXXAL

SEQ ID NO: 29 - protein, 11 aa, synthetic construct
  VARIANT 11: note = L or E
XXLXXILXXAL

SEQ ID NO: 30 - protein, 10 aa, synthetic construct
GGSWHHHHHH

SEQ ID NO: 31 - protein, 33 aa, synthetic construct
TXXXAXXXAXXTXXXAXXXAXXTXXXAXXXAXX

SEQ ID NO: 32 - protein, 44 aa, synthetic construct
TXXXAXXXAXXTXXXAXXXAXXTXXXAXXXAXXTXXXAXXXAXX